(19) United States
(12) Patent Application Publication
(10) Pub. No.: US 2020/0215114 A1
(43) Pub. Date: Jul. 9, 2020

(54) COMPOSITIONS AND METHODS FOR PRODUCING EXOSOME LOADED THERAPEUTICS FOR THE TREATMENT OF MULTIPLE ONCOLOGICAL DISORDERS

(71) Applicant: Exosome Therapeutics, Inc., San Diego, CA (US)

(72) Inventors: Gerardo Rodriguez-Araujo, San Diego, CA (US); Stephen R. Puckett, SR., Charlotte, NC (US); Stephen R. Puckett, JR., Charolotte, NC (US); Mitchell W. Puckett, Charolotte, NC (US)

(73) Assignee: Exosome Therapeutics, Inc., San Diego, CA (US)

(21) Appl. No.: 16/688,944

(22) Filed: Nov. 19, 2019

Related U.S. Application Data
(63) Continuation-in-part of application No. 16/591,502, filed on Oct. 2, 2019.
Application No. 16/591,502 is a continuation-in-part of application No. 16/591,483, filed on Oct. 2, 2019.
(60) Provisional application No. 62/769,123, filed on Nov. 19, 2018.
(60) Provisional application No. 62/769,774, filed on Nov. 20, 2018.
(60) Provisional application No. 62/769,711, filed on Nov. 20, 2018.
(60) Provisional application No. 62/770,640, filed on Nov. 21, 2018.

(51) Int. Cl. A61K 35/17; C07K 14/705; C12N 9/22; C12N 15/86

(52) U.S. Cl. CPC A61K 35/17; C07K 14/705; C12N 2310/20; C12N 15/86; C07K 2319/81; C12N 9/22

(57) ABSTRACT

A composition for delivering cargo to cytoplasm of a cell, wherein the cargo treats oncological disorders. In one embodiment the composition comprises: an exosome; and cargo, located within the exosome, comprising at least one plasmid. In another embodiment the composition comprises: an exosome; and cargo, located within the exosome, comprising a at least one plasmid. Wherein the cargo transduces autologous T cells into Chimeric Antigen Receptor T cells (CAR-T cells), which comprise at least one antigenic target.

### CROSS-REFERENCE TO RELATED APPLICATION

The present application is a continuation-in-part of, and claims priority to, U.S. patent application Ser. No. 16/591,502 filed Oct. 2, 2019, entitled “EXOSOME LOADED THERAPEUTICS FOR TREATING SICKLE CELL DISEASE,” which claims priority to U.S. Provisional Patent Application Nos. 62/740,396 filed Oct. 2, 2018, entitled “METHODS OF PRODUCING cGMP GRADE AND RESEARCH ONLY GRADE AUTOLOGOUS AND ALLOGENIC EXOSOMES AS CARRIERS FOR THERAPEUTIC COMPOUNDS FOR USE IN HUMANS AND IN PRECLINICAL STUDIES IN ANIMALS;” and 62/769,123 filed Nov. 19, 2018, entitled “cGMP Exosome Loaded Therapeutics for Sickle Cell Disease (SCD), SCD Anemia and its Associated Complications Reverting the Single Gene Mutation from Thymine to Adenine in the SNP rs334 in the Chromosome 11 and/or Reestablishing Normal Wild Type Healthy Genotype T>A (normal Adenine phenotype) to Produce Adult Beta Globin for Use in Humans and in Preclinical Studies in Animals,” the entire disclosures or which are incorporated by reference herein.

The present application also claims priority to, U.S. patent application Ser. No. 16/591,483 filed Oct. 2, 2019, entitled “COMPOSITIONS AND METHODS FOR PRODUCING EXOSOME LOADED THERAPEUTICS FOR TREATING CARDIOVASCULAR DISEASE,” which claims priority to U.S. Provisional Patent Application Nos. 62/740,396 filed Oct. 2, 2018, entitled “METHODS OF PRODUCING cGMP GRADE AND RESEARCH ONLY GRADE AUTOLOGOUS AND ALLOGENIC EXOSOMES AS CARRIERS FOR THERAPEUTIC COMPOUNDS FOR USE IN HUMANS AND IN PRECLINICAL STUDIES IN ANIMALS;” 62/740,382 filed Oct. 2, 2018, entitled “cGMP GRADE AND RESEARCH ONLY GRADE AUTOLOGOUS EXOSOMES FOR THE PROMOTION (USING VASCULAR ENDOTHELIAL GROWN FACTOR 1, 2 AND 3) OR REPRESSION OF ANGIOGENESIS (ANTI-MIR-214) FOR USE IN HUMANS AND IN PRECLINICAL STUDIES IN ANIMALS;” and 62/740,391 filed Oct. 2, 2018, “cGMP GRADE AND RESEARCH ONLY GRADE AUTOLOGOUS EXOSOMES FOR THE PRIMARY AND/OR SECONDARY PREVENTION OF CARDIOVASCULAR DISEASE (CVD), INFLAMMATION AND THEIR ASSOCIATED COMPLICATIONS USING PCSK-9, IL-1B, IL4 AND 13 AS THERAPEUTIC TARGETS FOR USE IN HUMANS AND IN PRECLINICAL STUDIES IN ANIMALS,” the entire disclosures of which are incorporated by reference herein

The present application further claims priority to U.S. Provisional Patent Application Nos. 62/769,123 filed Nov. 19, 2018, entitled “cGMP Exosome Loaded Therapeutics for Sickle Cell Disease (SCD), SCD Anemia and its Associated Complications Reverting the Single Gene Mutation from Thymine to Adenine in the SNP rs334 in the Chromosome 11 and/or Reestablishing Normal Wild Type Healthy Genotype T>A (normal Adenine phenotype) to Produce Adult Beta Globin for Use in Humans and in Preclinical Studies in Animals;” 62/770,640 filed Nov. 21, 2018, entitled “CGMP EXOSOME LOADED THERAPEUTICS FOR THE TREATMENT OF MULTIPLE ONCOLOGICAL DISORDERS AND THE METHODOLOGY TO DESIGN, PRODUCE AND MANUFACTURE EXOSOME-BASED CAR-T CELLS FOR USE IN HUMANS AND IN PRECLINICAL STUDIES IN ANIMALS,” 62/769,774 filed Nov. 20, 2018, entitled “cGMP Exosome Loaded Therapeutics Using Depletion and Self-production of Antibodies Against Sodium Glucose Like Transporters 1 and/or 2 for the Treatment of Diabetes Mellitus Type 1 and Type 2, Non Alcoholic Steatosis, Non Alcoholic Steatohepatitis, Atherosclerotic Cardiovascular Disease and Chronic Heart Failure with Low and Preserved Ejection Fraction for Use in Humans and in Preclinical Studies in Animals,” and 62/769,711 filed Nov. 20, 2018 entitled “cGMP Exosome Loaded Therapeutics to Correct SERPINA1 Mutation and to Reestablish Normal Physiological Alpha 1 Antitrypsin Levels for Primary and Secondary Prevention of Alpha 1 Antitrypsin Deficiency Related-Liver Disease (including Emphysema and Cirrhosis), Lung Function Deficiencies and Chronic Obstructive Pulmonary Disease (COPD) for Use in Humans and in Preclinical Studies in Animals,” the entire disclosures of which are incorporated by reference herein.

### BACKGROUND OF THE INVENTION

### 1. Field of Invention

This invention relates to extracellular vesicles for therapeutic delivery, and more specifically, to compositions and methods of producing exosomes, comprising therapeutics for the treatment of multiple oncological disorders for use in humans and in preclinical studies in animals.

### 2. Description of Related Art

Oncological disorders have a significant impact in not only patients but also in the families of patients who require healthcare assistance, support, and economical resources. Oncological diseases remain one of the leading causes of death worldwide. It has been postulated that oncological diseases are the result of a deficient adaptive mechanism to the constantly evolving environment in not just humans, but other species in general (Pepper J W, Scott Findlay C, Kassen R, Spencer S L, Maley C C. Cancer research meets evolutionary biology. EvolApp 2009; 2(1): 62-70).

A variety of organs or biological systems can be affected through these adaptive mechanisms and classifying the oncological conditions depend on where the illnesses develop first, the severity and/or aggressiveness, and the embryonic cell lineage from which the illnesses derive. One of the main challenges in the oncology field is to design and/or identify treatments that suppress cancer cells while maintaining normal cells. Cancer cells express cell surface markers associated with a given cancer but also express other normal markers on their cell surface, which makes distinguishing cancer cells from normal cells very difficult. Conventional cytotoxic treatment, including chemotherapy and radiotherapy, has a limited effect on advanced oncological disorders due to the heterogeneity of cancer cells, which can cause adverse symptoms in response to treatment. In recent years, a novel immunological approach that utilizes chimeric antigen receptor T (CAR-T) cells have been used. Chimeric antigen receptors (CARs) are receptor proteins that have been engineered to give T cells the new ability to target a specific protein. T cells that have been genetical engineered to produce an artificial T-cell receptor are known CAR-T cells. CAR-T cell therapy uses T cells engineered with CARs for cancer therapy. The use of CAR-T cells therapy has given promise on not just depleting tumors or cancers, but eradicating them using a patient's own immune T cells to attack and kill cancer cells while leaving normal cells intact (Biswas M, Kumar S R P, Terhorst C, Herzog R W. Gene therapy with Regulatory T Cells: a Beneficial Alliance. Front Immunol 2018; 9:554).

However, there are several limitations when using the CAR-T cell methodology. For example, the CAR-T cell methodology often uses viral vectors to bioengineer the T cells to include specific epitopes, recognition sites, or other cell surface markers in order to target specific cancer cells (Miliotou A N, Papadopoulou L C. CAR T-Cell Therapy: A New Era in Cancer Immunotherapy. Curr Pharm Biotechnol 2018; 19(1):5-18). Such viral vectors can trigger immunological responses in the transduced T cells and generate neutralizing antibodies once injected back into a patient. Another major issue in clinical trials is the viral genome integration into the host DNA with this viral vector technology (Mingozzi F. AAV Immunogenicity: A Matter of Sensitivity. Mol Ther 2018: 26(10): 2335-6). In addition, viral vectors such as adeno-associated viruses (AAV), adenoviruses (AdV), or any other viruses used for drug delivery do not meet the same bioequivalence requirement and there is significant variation in purity, stability, and efficacy from one lot to another. This poses challenging adjustments not only in dosing but also in safety and efficacy endpoints. (Colella P, Ronzitti G, Mingozzi F. Emerging Issues in AAV-Mediated in vivo Gene Therapy. Mol Ther Methods Clin Dev 2018; 8: 87-104).

Current drug developers face challenges in using viral vectors to deliver target genetic materials to cells and tissues due to the side effects of viral transfection and eliciting undesired immune responses. Both viral and non-viral vectors have shown modest to poor results, with the caveat of inducing immune responses. Specifically, existing viral and non-viral vectors generate antibodies, anti-viruses, or anti-carriers that limit the bioavailability and decrease the safety profile of a potential therapeutic product (Arrighetti N, Corbo C, Evangelopoulos M, Pasto, A, Zuco V, Tasciotti E. Exosome-like nanovectors for drug delivery in cancer. Curr Med Chem 2018; Khan M S and Roberts M S. Challenges and innovations of drug delivery in older age. Adv Drug Delivery Rev 2018).

Extracellular vesicles called exosomes are endogenous particles found in all body compartments that are highly effective and efficient in cell communication (Arrighetti N, Corbo C, Evangelopoulos M, Pasto, A, Zuco V, Tasciotti E. Exosome-like nanovectors for drug delivery in cancer. Curr Med Chem 2018). In particular, exosomes exist in body fluids such as blood, urine, and biological secretions. The function of exosomes is to share information between cells in a rapid and efficient manner. This cell-to-cell communication facilitates delivery and receipt of information (e.g., genetic materials, proteins, particles, signals, etc.), which allows specific cellular microenvironments to synchronize their function and their architecture in response to any stimulus. Exosomes are relatively small and flexible particles usually between 30 to 130 nanometers in diameter and are composed of similar materials to normal endogenous cell membranes. Hence, exosomes are highly effective and well-tolerated with minimal to no adverse effects, as a natural cell communication pathway for cells to share information among cells. Genetic material can be inserted into an exosome to be delivered to nearby or distant cells. Exosomes have the advantages of cell transduction up to 100% with high efficiency and fidelity, non-viral and non-immunogenic effects, enabling long transgenes, RNA, proteins, etc. Exosomes represent a safe, non-viral, drug delivery system in vivo to nearby or distal cells for treating disease, including multiple oncological disorders.

In light of the described challenges, there is an unmet medical need for an improved drug delivery system using exosome-based therapeutics or diagnostics in humans. In particular, there is a need for current good manufacturing practices (cGMP) exosome-based therapeutics to better target cancer cells through the use of CAR-T cells in order to treat multiple oncological disorders in vivo.

### SUMMARY OF THE INVENTION

The present invention overcomes these and other deficiencies of the prior art by providing cGMP autologous and/or universal donor exosomes loaded with cGMP grade genetic materials in order to treat multiple oncological disorders. The present invention describes exosome-mediated compositions that treat multiple oncological disorders by transducing autologous T cells into CAR-T cells with antigenic targets.

In an embodiment of the invention, cGMP grade autologous exosomes are loaded with cargo that comprises AdV, AAV, retrovirus, lentivirus, or a combination thereof that transduces T cells to express the CAR that recognizes specific antigenic markers such as CD19 and other cell surface markers in specific cancer categories. The CAR-T cells are then infused back into the patient and therapeutic outcomes are assessed.

In certain embodiments, the compositions and methods of using exosomes are used to treat cancer and oncological disorders including carcinogenesis, malignancies, tumors, metastasis, nodules of any variety (Endodermal, Mesodermal, or Ectodermal origin and due to spontaneous mutations or Human Papilloma Virus (HPV) or other viral infections), additional indications, cell therapeutics, vector and cell engineering, pharmacology, and toxicology assay development, immunological diseases and autoimmune diseases, rare diseases, etc.

In an embodiment, the invention improves the ability to transduce T-cells into CAR-T cells by reducing immune responses in T cells in response to viral vectors and plasmids allowing for reduced health care costs, increased the efficacy of treatment through exosome delivery, and limited immune responses from therapeutic delivery.

The foregoing, and other features and advantages of the invention, will be apparent from the following, more particular description of the preferred embodiments of the invention, the accompanying drawings, and the claims.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 illustrates a method of producing autologous exosomes from a body fluid according to an embodiment of the invention.

FIG. 2 illustrates a method of producing autologous exosomes according to another embodiment of the invention.

FIG. 3 illustrates a method of producing allogenic exosomes from a cell culture according to an embodiment of the invention.

FIG. 4 illustrates a method of producing allogenic exosomes from a body fluid according to another embodiment of the invention.

FIG. 5 illustrates a table of parameters for exosome isolation and purification according to an embodiment of the invention.

FIG. 6 illustrates a table of oncological targets in CAR-T cells according to multiple embodiments of the invention.

FIG. 7 illustrates transfection using exosomes loaded with exosomal cargo according to an embodiment of the invention.

FIG. 8 illustrates self-production of monoclonal neutralizing antibodies against the active sites of cancer cell surface markers using a plasmid.

FIG. 9 illustrates an exosome loaded with cargo according to an embodiment of an invention.

FIG. 10 illustrates an exosome loaded with cargo according to an embodiment of an invention.

### DETAILED DESCRIPTION OF PREFERRED EMBODIMENTS

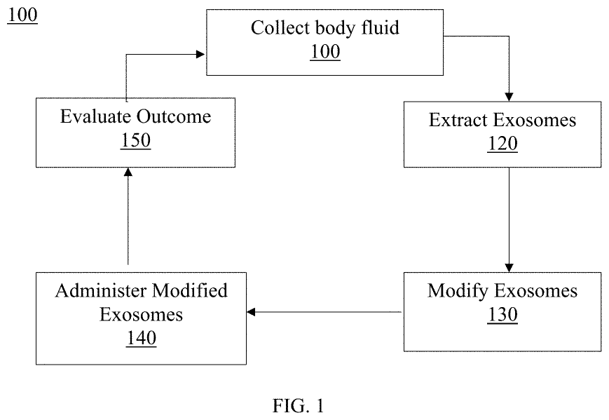

Preferred embodiments of the present invention and their advantages may be understood by referring to FIGS. 1-10. The described features, structures, or characteristics of the invention may be combined in any suitable manner in one or more embodiments. It will be apparent to those skilled in the art that various modifications and variations can be made to the present invention without departing from the spirit and scope of the invention. Thus, it is intended that the present invention cover the modifications and variations of this invention provided they come within the scope of the appended claims and their equivalents.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Moreover, the described features, structures, or characteristics of the invention may be combined in any suitable manner in one or more embodiments. It will be apparent to those skilled in the art that various modifications and variations can be made to the present invention without departing from the spirit and scope of the invention. Thus, it is intended that the present invention cover the modifications and variations of this invention provided they come within the scope of the appended claims and their equivalents. Reference will now be made in detail to the preferred embodiments of the invention.

The term “exosome” as used herein refers to any extracellular vesicle derived from any body fluid from a human or an animal (e.g., blood), any extracellular vesicle derived from human or animal cell lines, cell cultures, and primary cultures not limited to autologous exosomes, universal donor exosomes, allogenic exosomes, and modified exosomes. In certain examples, the exosome is made to meet pharmaceutical and cGMP standards.

The term “cargo” as used herein refers to any type of molecule or any type of RNA including miRNA, mRNA, tRNA, rRNA, small interfering RNA (siRNA), RNAi, regulating RNA, gRNA, long interference RNA, non-coding and coding RNA; any type of DNA including DNA fragments, DNA plasmids, interference DNA (DNAi); including any type of nucleic acid including antisense oligonucleotides (ASO); any genetic material; any genetic construct; any nucleic acid construct; any plasmid or vector; any protein including recombinant endogenous protein, enzyme, antibody, wnt signaling proteins; any lipid; any therapeutic molecule or diagnostic molecule; any cellular component; CAR-T cell transduced without using retroviruses; any virus including retrovirus, AdV, AAV of any variety and strain, and DNA viruses; any gene editing technology including clustered regularly interspaced short palindromic repeats (CRISPR), CRISPR/CAS9 system, any endonucleases for base editing, a Zinc finger, a single base editor, Transcription activator-like effector nucleases (TALENs), any meganuclease; any synthetic molecular conjugate; or combination thereof loaded into an exosome. Typically, such cargo is naturally not present in the exosome. In certain examples, the cargo is made to meet pharmaceutical and cGMP standards.

In one embodiment cargo could include a promoter. The term “promoter” as used herein refers to any DNA sequence that promotes the transcription of a gene. A plasmid comprises a tissue-specific promoter. Moreover, the promoter comprises any tissue-specific promoter (e.g., lung, liver, or any other tissue type), a self-inactivating (SIN) sequence, vesicular stomatitis virus-G protein (VSV-G), or a combination thereof. The advantage of using a tissue-specific promoter is to better target a desired tissue in which to transcribe RNA and subsequently encode a protein.

The term “fluid” as used herein refers to any type of body fluid produced by a human or an animal including but not limited to blood, cerebral spinal fluid, urine, saliva, and any biological secretions, etc.

FIGS. 1-4 illustrate methods of producing exosomes and cargo, and methods for cargo loading into exosomes. Such improved methods and techniques would be appreciated by one of ordinary skill, especially those for increasing yield of purified exosomes and efficient loading of exosome cargo for use in preclinical and clinical studies. The methods of loading genetic material (e.g., constructs of DNA or RNA, or any type of nucleic acids) directly into exosomes are transformation, transfection and microinjection. In one embodiment, exosomes are extracted, isolated and purified from peripheral blood mononuclear cells (PBMC) circulating in peripheral blood. In such an embodiment, PBMCs are harvested from a patient or a universal donor. PBMCs are isolated and expanded in vitro using closed systems for cell culture. In another embodiment, open systems may be used depending on available resources. PBMCs produce and secrete exosomes into the media of a cell culture. The media can be filtered and exosomes can be sorted by specific parameters and purified to improve exosome quality.

The present extracellular vesicular compositions may be used to treat any of the following diseases including, but not limited to: 1. Cancer and oncological disorders including carcinogenesis, malignancies, tumors, metastasis, nodules of any variety (endodermal, mesodermal or ectodermal origin and due to spontaneous mutations or human papillomavirus or other viral infections); 2. Infectious diseases including human immunodeficiency virus and Ebola viral infections; 3. Cardiovascular disease including coronary arterial disease, peripheral vascular disease, peripheral arterial disease, chronic heart failure (ischemic and non-ischemic), stroke, acute kidney failure, endothelial dysfunction, mitochondrial dysfunction, oxidative stress, etc.; 4. Diabetes mellitus including Type-1 diabetes mellitus and Type-2 diabetes mellitus and any of related complications such as diabetic foot, diabetic retinopathy, peripheral diabetic neuropathy, diabetic kidney disease, insulin resistance, pre-diabetes, gestational diabetes, etc.; 5. Non-alcoholic liver disease, non-alcoholic steatohepatitis, non-alcoholic cirrhosis for primary and secondary prevention; 6. Obesity, overweightness, obesity type-1, type-2, and type 3, morbid obesity, and bariatric surgery; 7. Rare diseases; 8. Gastro-intestinal diseases including Ulcerative colitis, Crohn's disease, etc.; and 9. Musculo-skeletal diseases. Further, the present exosome compositions may be used for cell therapeutics, vector and cell engineering, pharmacology and toxicology assay development, and similar such processes.

The present invention relates to using modified exosomes loaded with cargo for treatment of multiple oncological disorders.

FIG. 1 illustrates a method for producing autologous exosomes from a body fluid according to an embodiment of the invention. Although the method 100 is illustrated and described as a sequence of steps, its contemplated that various embodiments of the method 100 may be performed in any order or combination and need not include all of the illustrated steps. The method 100 comprises the step of: collecting body fluid 110 from a subject, extracting exosomes 120 from the body fluid, modifying said exosomes 130, administering modified exosomes 140, and evaluating a health-related outcome 150.

In step 110, body fluid is collected from a subject. The subject may be a human or an animal. The body fluid can be peripheral blood, cerebral spinal fluid, secretions, or any other body fluid in which exosomes can be extracted.

In step 120, exosomes are extracted from the body fluid. The extraction method depends on a number of factors including the type of body fluid extracted. Peripheral blood, for example, contains peripheral blood mononuclear cells and cellular component layers that can be separated by centrifugation at a medical facility. During the extraction process plasma, cells and cellular components are kept on dry ice at all times before isolation.

In one embodiment, the body fluid is transported to a laboratory to undergo isolation. In one embodiment, exosome isolation is achieved using a gradient method or a designated isolation kit (i.e., Total Exosome Isolation kit, ThermoFisher). The isolation kit protocol is highly efficient in yielding high amounts of exosomes from a body fluid, a cell culture media, or cell.

The method 100, provides several approaches to further optimize isolation of exosomes and increase exosome yield from a body fluid. In one embodiment, a gradient column separates components of the collected peripheral blood by cell densities. Such cellular densities correspond to exosomes and exosome-related materials. Another embodiment uses an exosome sorting method, where sorting markers or sorting beads are used to isolate exosomes from solution. A further embodiment uses flow cytometry sorting, which uses surface biomarkers present on exosome to identify and sort exosomes and exosome-related materials from cells and cell suspensions. In one embodiment, an exosome can be modified to include a targeting agent on a surface of the exosome.

Specifically, the exosomes can be modified (modified exosomes) to have specific targeting agents on their surface. In various examples, the modified exosome may have a targeting agent covering an entire surface or a partial surface of the extracellular vesicle. Thin layer chromatography can be used to optimally separate exosomes and exosome-related products according to specific exosome-associated surface proteins and lipids. An exosome from peripheral blood, for example, would have exosome-related products such as transferrin receptors (immature exosomes), signaling molecules, and similar cellular components. In another embodiment, ionic separation by drift time can be used to optimize isolating exosomes. For example, mass spectrometry may be used to isolate high yields of exosome and exosome-related products on the order of. Ion mobility spectrometry-mass spectrometry may also be performed when physicochemical properties of both the exosome and the cargo need to be defined prior to loading into the exosome.

Isolated exosome samples can be purified using column methods in accordance with cGMP protocols and regulatory requirements.

In step 130, the exosomes are modified by incorporating cargos. In one embodiment, the modifications to the exosomes are done ex vivo. The exosomes can be further modified to have specific protein epitopes on their surfaces. Exosomes are assembled or transfected with cargo using a number of methods. In one embodiment depending on the physicochemical properties of the load, the exosomes are assembled or transfected with cargo using liposomes (Lipofectamine 2000, Exofect, or heat shock). In another embodiment, exosomes are assembled or transfected with cargo using CAR-T cells transduced without using retroviruses. In another embodiment, exosomes are assembled or transfected with cargo using retroviruses, AdV, AAV of any variety and strain. In another embodiment, exosomes are assembled or transfected with cargo using DNA viruses, siRNA, long interference RNA, noncoding RNA, RNAi, RNA vectors. In another embodiment, exosomes are assembled or transfected with cargo using DNA, DNA plasmids, CRISPR, CRISPR/CAS9 and/or any endonucleases for gene editing. In another embodiment, exosomes are assembled or transfected with cargo using gene editing technology, small molecules, antibodies, and proteins including recombinant endogenous proteins. In another embodiment, exosomes are assembled or transfected with cargo using oligonucleotide therapeutics, including ASO, gene targeting technology, and gene correction technology. In another embodiment, exosomes are assembled or transfected with cargo using synthetic/molecular conjugates and physical methods for delivery of gene and cell therapeutics.

In step 130, the method for loading exosomes efficiently and effectively incorporates autologous or exogenous materials (therapeutic compounds above or any endogenous enzyme, protein, lipid, molecule, DNA or RNA of interest). In non-limiting examples, the method for loading an exosome can include the process of: 1) Lipid-lipid affinity, using liposomes of high and low density; 2) Incorporating intracellular affinity proteins and/or molecules into the exosome; 3) Using Clathrin coated vesicles in clathrin-mediated endocytosis methods for incorporation of a therapeutic molecule into an exosome or an exosome-like carrier; and 4) Endocytosis receptors/proteins methodology. In one embodiment the method for loading exosomes includes the methods of exosome membrane dissociation and reconstitution via chemical or electromagnetic gradient changes. In one embodiment, a method is used for large molecules or heavy compounds. In certain examples, the optimization of the method 100 is due to including transmembrane transporters activators when loading the biological materials into the exosomes. After the exosome has been loaded, any potential activator remaining in the exosome will be filtered and purified using column methods in compliance with cGMP and regulatory requirements before undergoing the next processing steps.

Exosomes loaded with cargo are considered mature exosomes and are inspected for cGMP compliance, purity and stability for quality assurance and quality check. Next, mature exosomes that have passed the quality check undergo an expansion process if needed. Next, the mature exosomes are diluted and premix into saline/vehicle (depending on the characteristics of the load) for a ready to administer tube/cartridge. Finally, the suspension is frozen and stored or shipped to a site for use in clinical or preclinical studies and to patients for self-injection of approved-clinical grade mature exosomes.

In step 140, the mature exosomes are administered to a subject. The subject may be the same subject from which the body fluid was collected in step 110. The method of administering the exosomes 140 includes, but is not limited to: intravenous, intra-arterial, intrathecal, intraventricular, subcutaneous, subdermal, oral, rectal, intraperitoneal, transdermal, intraosseous injection, intraosseous infusion, or a combination thereof. In one embodiment the mature exosomes are administered in vivo.

In step 150, the outcome of the treatment is evaluated. This evaluation can be done using a variety of different methods, which is immediately apparent to one of ordinary skill in the art.

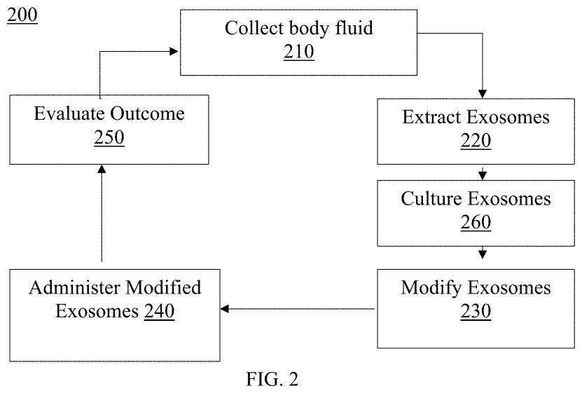

FIG. 2 illustrates a method for producing autologous exosomes from a body fluid according to an embodiment of the invention. Although the method 200 is illustrated and described as a sequence of steps, its contemplated that various embodiments of the method 200 may be performed in any order or combination and need not include all of the illustrated steps. The method 200 comprises the step of: collecting body fluid 210 from a subject, extracting exosomes 220 from the body fluid, culture the exosomes 260, modifying the exosomes 230, administering modified exosome 240, and evaluating the outcome 250.

In step 210, body fluid is collected from a subject. The subject may be a human or an animal. The body fluid can be peripheral blood, cerebral spinal fluid, secretions, or any other body fluid in which exosomes can be extracted.

In step 220, exosomes are extracted from the body fluid using methods as explained above.

In step 260 the exosomes are subjected to a primary culture and expansion. The exosomes will be extracted from primary cultured cells using a gradient or filtration method or a designated expansion kit (i.e., Total Exosome Isolation kit (from cell culture media), ThermoFisher). The cell culture and expansion may be frozen and stored for future exosome isolation procedures/protocols per the methods described above.

In step 230, the exosomes are modified by incorporating cargos. Exosomes are assembled or transfected with cargo using a number of methods as explained above. In one embodiment, the step of modifying the exosomes occurs ex vivo.

In step 240 the mature exosomes are administered to a subject using methods as explained above. The step of administering the modified exosomes can occur either in vivo or in vitro.

In step 250, the outcome of the treatment is evaluated. This evaluation can be done using a variety of different methods, which is immediately apparent to one of ordinary skill in the art.

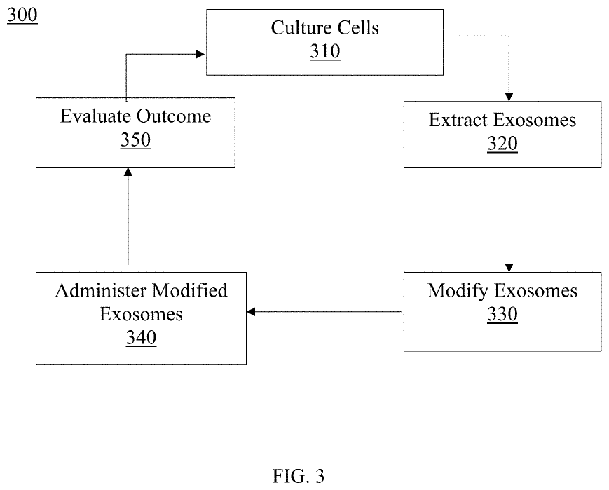

FIG. 3 illustrates a method for producing autologous exosomes from a cell culture according to an embodiment of the invention. Although the method 300 is illustrated and described as a sequence of steps, its contemplated that various embodiments of the method 300 may be performed in any order or combination and need not include all of the illustrated steps. The method 300 comprises the step of: culturing cells 310, extracting exosomes 320 from the cell culture, modifying the exosomes 330, administering modified exosome 340, and evaluating the outcome 350.

In step 310, primary or stable cell lines of human or animal origin are cultured and expanded with standard conditions.

In step 320, exosomes are extracted from the cultured cells.

In step 330, the exosomes are modified by incorporating cargos. Exosomes are assembled or transfected with cargo using a number of methods as explained above.

In step 340 the mature exosomes are administered to a subject using methods as explained above.

In step 350, the outcome of the treatment is evaluated. This evaluation can be done using a variety of different methods, which is immediately apparent to one of ordinary skill in the art.

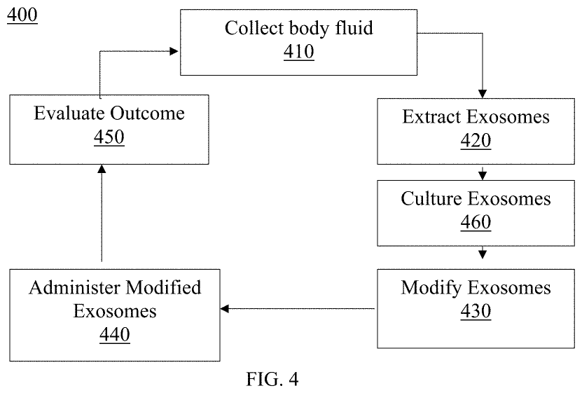

FIG. 4 illustrates a method for producing autologous exosomes from body fluid according to an embodiment of the invention. Although the method 400 is illustrated and described as a sequence of steps, it's contemplated that various embodiments of the method 400 may be performed in any order or combination and need not include all of the illustrated steps. The method 400 comprises the step of: collecting body fluid 410, extracting exosomes 420 from the body fluid, culturing the exosomes 460, modifying the exosomes 430, administering modified exosome 440, and evaluating the outcome 450.

In step 410, a body fluid is collected from a universal donor or patient. The subject may be a human or an animal. The body fluid can be peripheral blood, cerebral spinal fluid, secretions, or any other body fluid in which exosomes can be extracted.

In step 420, exosomes are extracted from the body fluid using methods as explained above.

In step 460, the exosomes are cultured. The exosomes are expanded using a primary cell culture from the body fluid of the universal donor or patient using a gradient method or a designated isolation kit (i.e., Total Exosome Isolation kit, ThermoFisher). The isolation kit protocol is highly efficient in yielding high amounts of exosomes from either body fluids or cell culture media or cell. The cell culture and expansion from the universal donor or patient may be frozen and stored for future exosome isolation procedures/protocols per the methods described above.

In step 430, the exosomes are modified by incorporating cargos. Exosomes are assembled or transfected with cargo using a number of methods as explained above.

In step 440 the mature exosomes are administered to a subject using methods as explained above.

In step 450, the outcome of the treatment is evaluated. This evaluation can be done using a variety of different methods, which is immediately apparent to one of ordinary skill in the art.

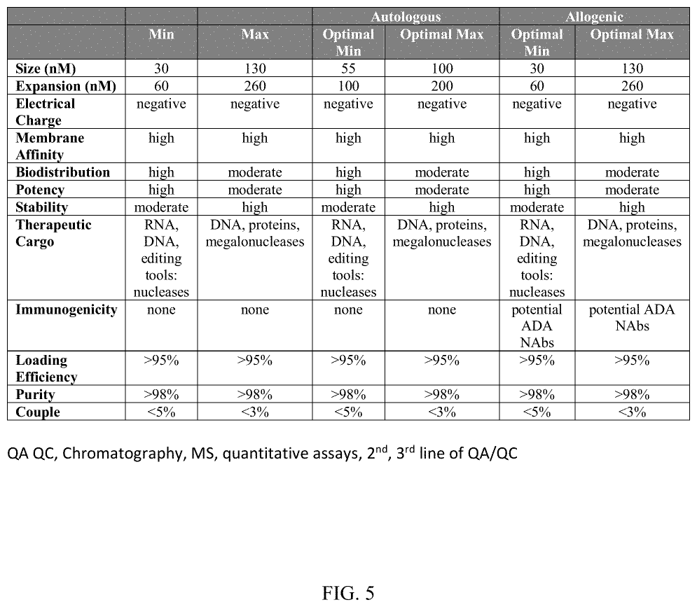

FIG. 5 illustrates the parameters used to sort exosomes according to an embodiment of the invention. In one embodiment, the invention provides autologous exosomes having an optimal vesicle size between about 55 nanometers (nM) and 100 nM. In certain embodiments, allogenic exosomes have an optimal vesicle size between about 30 nM and 130 nM. A vesicle size between 55 nM and 100 nM may be chosen as larger exosomes are less stable. Also, larger exosomes can couple with other exosomes making calculating drug dose, bioavailability, and biodistribution challenging. In some embodiments, the exosomes have the ability to expand to a size between about 60 nM and 260 nM. Such expanded exosomes can encompass large constructs. In some embodiments, the expanded exosomes can encompass more than or equal to about 7 kilo bases (Kb), and accommodate one or more copies of a relatively large viral particle such as an AAV. In one example, an exosome is loaded with at least four AAV particles to improve an exosome safety profile. In some embodiments, either an autologous or allogenic exosome has a negative electrical charge. Both autologous and allogenic exosomes can have a high membrane affinity. In some embodiments, biodistribution is moderate to high. Potency can range from high to moderate. Stability is moderate to high.

In an embodiment, exosomes can comprise a smaller sized cargo comprising RNA, DNA, editing tools (e.g., nucleases), or any combination thereof. In another embodiment, an exosome can comprise a larger cargo comprising DNA, proteins, megalonucleases, or a combination thereof. One advantage of autologous exosomes is that they do not illicit a significant immune response. Allogenic exosomes may illicit anti-drug antibodies (ADA) and neutralizing anti-bodies (NAb). One embodiment of the present invention enables high efficiency of loading cargo into at least ninety-five percent (95%) of exosomes. Another embodiment can provide a higher purity of exosomes of at least ninety-eight percent (98%).

Additionally, an exosome can further be modified to include a targeting agent on a surface of the exosome. For example, an exosome can have specific protein epitopes, plasma membrane components, etc.

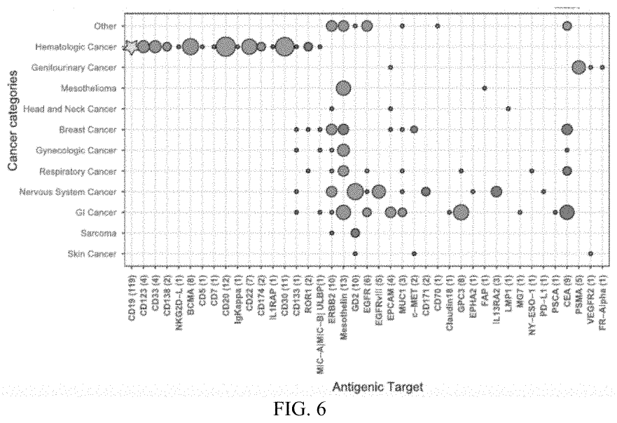

FIG. 6 illustrates a table of the oncological targets in CAR-T cells according to multiple embodiments of the invention. The table shows cancer categories on the y-axis and antigenic targets on the x-axis. The size of the circles in the table indicates the number of clinical trials for each antigenic target for a specific cancer with increasing size of circle corresponding to increasing number of clinical trials.

The present exosome compositions may be used to treat any of the following cancer categories including, but not limited to: 1. Hematologic cancer; 2. Genitourinary cancer; 3. Mesothelioma; 4. Head and neck cancer; 5. Breast Cancer; 6. Gynecologic cancer; 7. Respiratory cancer; 8. Nervous system cancer; 9. Gastrointestinal (GI) cancer; 10. Sarcoma; and 11. Skin Cancer.

The present exosome compositions may be used to transduce T cells into CAR-T cells with any of the following antigenic targets, but are not limited to: 1. CD19; 2. CD123; 3. CD33; 4. CD138; 5. NKG2D-L; 6. BCMA; 7. CD5; 8. CD7; 9. CD20; 10. IgKappa; 11. CD22; 12. CD174; 13. IL1RAP; 14. CD30; 15. CD133; 16. ROR1; 17. MIC-A/MIC-B/ULBP; 18. ERBB2; 19. Mesothelin; 20. GD2; 21. EGFR; 22. EDRvIII; 23. EPCAM; 24. MUC1; 25. C-MET; 26. CD171; 27. CD70; 28. Claudin18; 29. GPC3; 30. EPHA2; 31. FAP; 32. IL13RA2; 33. LMP1; 34. MG7; 35. NY-ESO-1; 36. PD-L1; 37. PSCA; 38. CEA; 39. PSMA; 40. VEGFR2; and 41. FR-Alpha.

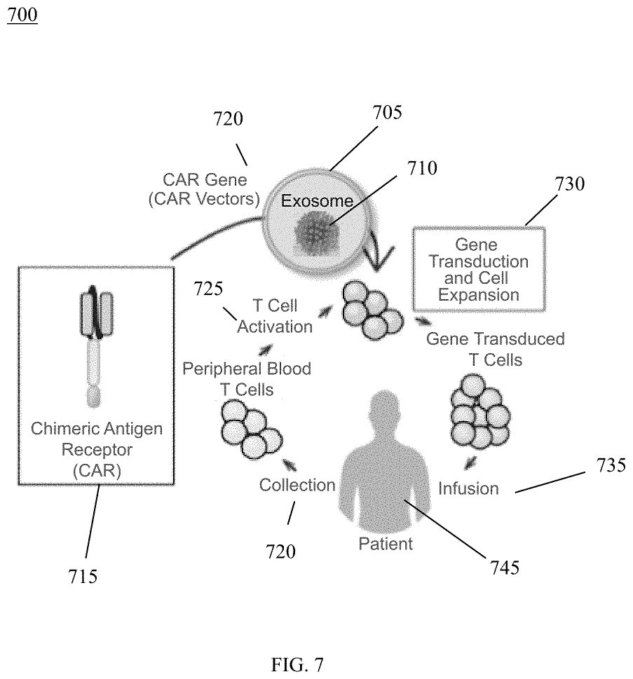

FIG. 7 illustrates a method 700 of transfection using an exosome 705 loaded with exosomal cargo 710 directed against antigens using CARs 715 according to an embodiment of the invention. Although the method 700 is illustrated and described as a sequence of steps, it's contemplated that various embodiments of the method 700 may be performed in any order or combination and need not include all of the illustrated steps. The exosomal cargo includes but is not limited to siRNA, plasmid DNA, proteins, antibodies, etc. In step 720, body fluid is collected from the patient 745 using techniques described above. In step 725, the T cells are activated. In an embodiment, exosomes are isolated from the body fluid which was collected from the patient. In another embodiment, the exosomes may be isolated from cultured cells. The exosomes are then modified as discussed above to include cargo. In step 730, the T cells are transduced into CAR-T cells and then expanded. In an embodiment, when a virus-free CAR-T cell design is desired, T cells can be isolated from the patient and expanded in a culture and/or bioreactor. In step 730 the gene transduced T cells are administered to the patient.

In another embodiment, the exosomal cargo 710 includes, but is not limited to, AdV, AAV, retrovirus, lentivirus, or any combination thereof.

In other embodiments, the exosomal cargo 710 includes viral and non-viral materials. In such an embodiment, the method of preparing CAR-T cells may have two modalities: CAR-T cell generation using exosomes with vectors of CARs and at least one antigenic target and CAR-T cell generation using exosomes to deliver viruses (virus loaded with CARs and epitope expression cassettes). In certain embodiments, the exosome is autologous or from a universal donor. In one embodiment, the exosome 705 comprises a cargo 710 that includes a retrovirus to express a specific CAR gene.

The present exosome compositions may be used to treat any of the following cancer categories including, but not limited to: 1. Hematologic cancer; 2. Genitourinary cancer; 3. Mesothelioma; 4. Head and neck cancer; 5. Breast Cancer; 6. Gynecologic cancer; 7. Respiratory cancer; 8. Nervous system cancer; 9. Gastrointestinal (GI) cancer; 10. Sarcoma; and 11. Skin Cancer.

The present exosome compositions may be used to transduce T cells into CAR-T cells with any of the following antigenic targets, but are not limited to: 1. CD19; 2. CD123; 3. CD33; 4. CD138; 5. NKG2D-L; 6. BCMA; 7. CD5; 8. CD7; 9. CD20; 10. IgKappa; 11. CD22; 12. CD174; 13. IL1RAP; 14. CD30; 15. CD133; 16. ROR1; 17. MIC-A/MIC-B/ULBP; 18. ERBB2; 19. Mesothelin; 20. GD2; 21. EGFR; 22. EDRvIII; 23. EPCAM; 24. MUC1; 25. C-MET; 26. CD171; 27. CD70; 28. Claudin18; 29. GPC3; 30. EPHA2; 31. FAP; 32. IL13RA2; 33. LMP1; 34. MG7; 35. NY-ESO-1; 36. PD-L1; 37. PSCA; 38. CEA; 39. PSMA; 40. VEGFR2; and 41. FR-Alpha.

In one embodiment, CAR-T cells are prepared using a non-viral vector (exosomes) to transduce cells to express CAR recognizing specific markers such as CD-19 and/or other cell surface markers, molecules, or antigenic targets. Furthermore, this method can include bioengineering T cells using exosomes loaded with cargo, which can included, but is not limited to, non-viral expression vectors, naked/conjugated mRNA or sleeping beauty transposons, etc. In such an embodiment, the exosomes deliver the cargo directly into the cytoplasm or nucleus of the T cells, transducing the T cells to recognize specific epitopes or antigenic targets, which causes the T cells to recognize cancer cells.

In another embodiment, CAR-T cells are prepared using exosomes as a non-viral drug delivery system carrying viral vectors such as AdV, AAV, lentiviruses, and retroviruses to generate specific CARs. Some advantages of these type of exosome-based CAR-T cells include, but are not limited to, improving transfection efficacy, improving transgene expression, minimizing or preventing immune exposure, and/or minimizing or preventing immune reactions.

In another embodiment, CARs are engineered receptors that can graft an arbitrary specificity onto an immune effector cell, therefore exosome-based generation of CAR-T cells can be a pathway to generate or bioengineer quality and potent CAR-T cells while avoiding problems such as immunogenicity and transduction efficacy of the transgene/cargo compared to viral or other non-viral vectors. This method of generating highly specific CAR-T cells, that recognize epitopes or antigenic targets, which are subsequently purified and verified using the highest available standards including cGMP for cGMP grade production of CAR-T cells, allows for human administration or for the use in preclinical studies in animals.

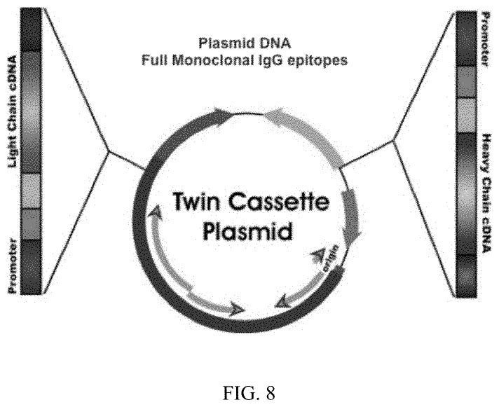

FIG. 8 illustrates self-production of monoclonal neutralizing antibodies against the active sites of cancer cell surface markers, as referenced above, using a plasmid loaded into an autologous or universal exosome. Such exosomes are prepared according to the methods described above in various embodiments. In some embodiments, an exosome can include another RNAi technology, GalNAc construct, a plasmid DNA, or a combination thereof. In an embodiment, when the exosome includes a DNA plasmid, the DNA plasmids may include doxycycline, ampicillin, kanamycin, another equivalent agent, or a combination thereof. In certain embodiments, the plasmid is used alone as monotherapy in preclinical and clinical trials as well as for human use. In one embodiment, the plasmid is delivered using exosomes to deliver either ex vivo to T cells to respond to a specific antibody. In another embodiment, the plasmid is delivered directly via intramuscular injection to illicit humoral agents to respond to a specific antibody.

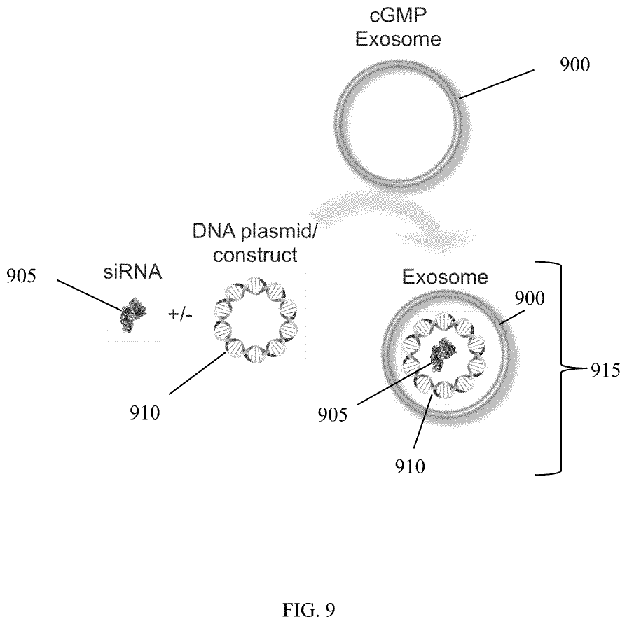

FIG. 9 illustrates a cGMP grade exosome 900 loaded with cargos according to an embodiment of an invention. The different methods for isolating and loading the cGMP exosome 900 have been described above. The cGMP exosome 900 is able to incorporate autologous or exogenous materials (therapeutic compounds above or any endogenous enzyme, protein, lipid, molecule, DNA or RNA of interest). In an embodiment, after the exosome 900 has been loaded, potential activator remaining in the exosome 900 may be filtered and purified using column methods in compliance with cGMP and regulatory requirements before undergoing the next processing steps. In an embodiment, the exosome 900 is loaded with a first cargo 905 and a second cargo 910. The first cargo 905 and the second cargo 910 may be plasmid secreting antibodies, wherein the plasmid is an RNA plasmid, an RNAi plasmid, a DNA plasmid, an DNAi plasmid, or a combination thereof. In one embodiment, the cGMP exosome 900 is loaded with cargo comprising a siRNA 905 and a DNA plasmid 910. The resulting mature exosome 915 is inspected for cGMP compliance, purity and stability for quality assurance and quality check. As discussed above, the mature exosomes, that have passed the quality check, may undergo an expansion process. In an embodiment, the mature exosomes are diluted and premix into saline or a similar such solution for a ready to administer tube. Finally, the suspension can be frozen and shipped to a site for use in clinical or preclinical studies and to patients for self-injection of approved-clinical grade mature exosomes.

The present exosome compositions may be used to treat any of the following cancer categories including, but not limited to: 1. Hematologic cancer; 2. Genitourinary cancer; 3. Mesothelioma; 4. Head and neck cancer; 5. Breast Cancer; 6. Gynecologic cancer; 7. Respiratory cancer; 8. Nervous system cancer; 9. Gastrointestinal (GI) cancer; 10. Sarcoma; and 11. Skin Cancer.

The present exosome compositions may be used to transduce T cells into CAR-T cells with any of the following antigenic targets, but are not limited to: 1. CD19; 2. CD123; 3. CD33; 4. CD138; 5. NKG2D-L; 6. BCMA; 7. CD5; 8. CD7; 9. CD20; 10. IgKappa; 11. CD22; 12. CD174; 13. IL1RAP; 14. CD30; 15. CD133; 16. ROR1; 17. MIC-A/MIC-B/ULBP; 18. ERBB2; 19. Mesothelin; 20. GD2; 21. EGFR; 22. EDRvIII; 23. EPCAM; 24. MUC1; 25. C-MET; 26. CD171; 27. CD70; 28. Claudin18; 29. GPC3; 30. EPHA2; 31. FAP; 32. IL13RA2; 33. LMP1; 34. MG7; 35. NY-ESO-1; 36. PD-L1; 37. PSCA; 38. CEA; 39. PSMA; 40. VEGFR2; and 41. FR-Alpha.

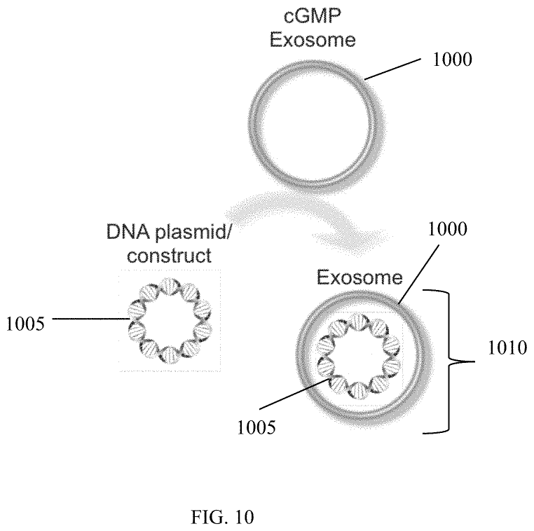

FIG. 10 illustrates an exosome 1005 loaded with cargo 1005 according to an embodiment of an invention. The different methods for isolating and loading the cGMP exosome 1000 have been described above. In one embodiment, the cGMP exosome 1000 is loaded with cargo 1005. The resulting mature exosome 1010 is inspected for cGMP compliance, purity and stability for quality assurance and quality check. As discussed above, the mature exosomes, that have passed the quality check, may undergo an expansion process. In an embodiment, the mature exosomes are diluted and premix into saline or a similar such solution for a ready to administer tube. Finally, the suspension can be frozen and shipped to a site for use in clinical or preclinical studies and to patients for self-injection of approved-clinical grade mature exosomes. In one embodiment the cargo 1005 is a DNA plasmid. In one embodiment, the cargo is a DNA plasmid expressing the antigenic target CD-19 to treat hematologic cancer.

The present exosome compositions may be used to treat any of the following cancer categories including, but not limited to: 1. Hematologic cancer; 2. Genitourinary cancer; 3. Mesothelioma; 4. Head and neck cancer; 5. Breast Cancer; 6. Gynecologic cancer; 7. Respiratory cancer; 8. Nervous system cancer; 9. Gastrointestinal (GI) cancer; 10. Sarcoma; and 11. Skin Cancer.

The present exosome compositions may be used to transduce T cells into CAR-T cells with any of the following antigenic targets, but are not limited to: 1. CD19; 2. CD123; 3. CD33; 4. CD138; 5. NKG2D-L; 6. BCMA; 7. CD5; 8. CD7; 9. CD20; 10. IgKappa; 11. CD22; 12. CD174; 13. IL1RAP; 14. CD30; 15. CD133; 16. ROR1; 17. MIC-A/MIC-B/ULBP; 18. ERBB2; 19. Mesothelin; 20. GD2; 21. EGFR; 22. EDRvIII; 23. EPCAM; 24. MUC1; 25. C-MET; 26. CD171; 27. CD70; 28. Claudin18; 29. GPC3; 30. EPHA2; 31. FAP; 32. IL13RA2; 33. LMP1; 34. MG7; 35. NY-ESO-1; 36. PD-L1; 37. PSCA; 38. CEA; 39. PSMA; 40. VEGFR2; and 41. FR-Alpha.

In some embodiments, base editors show low (0.1%) indel formation (insertion or deletion of bases in the genome), which makes it beneficial for therapeutic use. A nuclease base editor enables treatment of certain illnesses by targeting and correcting one or both alleles at a particular DNA sequence. In an embodiment, a single guide RNA (sgRNA) is designed and added to a nuclease base editor plasmid to increase precision on a target DNA sequence. In said embodiment, a protospacer, protospacer adjacent motif (PAM) sequence, and motifs surrounding a particular DNA sequence can be included in the target DNA sequence. Inclusion of a protospacer and a PAM sequence enable the CRISPR-CAS9 system to cleave the target DNA sequence. In such an embodiment, the expression plasmid with sgRNA can be cloned. Further, the sgRNA and the nuclease base editor can then be loaded into an exosome. In such an embodiment, a nuclease base editor corrects one or both alleles at a particular DNA sequence.

In another embodiment, the proportion of loading is 1:1 (exosome: base editor) using techniques that include electromagnetism and membrane dissociation technologies. In one embodiment, exosomes having a vesicle size between fifty-five (55) and one hundred (100) nM are selected for cargo loading.

FIGS. 9-10 illustrate exosomes loaded with different types of cargo. In an embodiment, any number of cargos discussed may be loaded into a single exosome. Further, the different types of cargo may be loaded into exosomes in any number of combinations. In one embodiment, the exosome may have two or more cargos wherein the two or more cargos may be identical or substantially the same. In another embodiment, an exosome may have two or more cargos wherein each of the two or more cargos are distinct from one another.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

1. A composition for delivering cargo to the cytoplasm of a cell, wherein the cargo treats oncological disorders, the composition comprising:
an exosome; and
cargo, located within the exosome, comprising at least one plasmid.

2. The composition of claim 1, wherein the cargo transduces autologous T cells into Chimeric Antigen Receptor T cells (CAR-T cells).

3. The composition of claim 1, wherein the CAR-T cells comprise at least one antigenic target.

4. The composition of claim 3, wherein the at least one antigenic target is CD19, CD123, CD33, CD138, NKG2D-L, BCMA, CD5, CD7, CD20, IgKappa, CD22, CD174, IL1RAP, CD30, CD133, ROR1, MIC-A/MIC-B/ULBP, ERBB2, Mesothelin, GD2, EGFR, EDRvIII, EPCAM, MUC1, C-MET, CD171, CD70, Claudin18, GPC3, EPHA2, FAP, IL13RA2, LMP1, MG7, NY-ESO-1, PD-L1, PSCA, CEA, PSMA, VEGFR2, FR-Alpha, or a combination thereof.

5. The composition of claim 4, wherein the exosome is isolated from autologous cells of a patient.

6. The composition of claim 4, wherein the exosome is isolated from a cell line, a primary cell culture, or a combination thereof.

7. The composition of claim 4, wherein the exosome is isolated from a stem cell.

8. The composition of claim 4, wherein the at least one plasmid is an RNA plasmid, a DNA plasmid, a retrovirus, adeno-associated virus (AAV), adenovirus (AdV), lentivirus, or a combination thereof.

9. The composition of claim 4, wherein the at least one plasmid further comprises a promoter.

10. The composition of claim 4, wherein the cargo further comprises a CRISPR-CAS9 system, a Zinc finger, a single base editor, or a combination thereof.

11. The composition of claim 4, wherein the at least one plasmid is a DNA plasmid bioengineered specifically to self-produce monoclonal neutralizing antibodies.

12. The composition of claim 4, wherein the exosome further comprises at least one targeting agent, protein epitope, or a combination thereof.

13. A composition for delivering cargo to the cytoplasm of a cell, wherein the cargo treats oncological disorders, the composition comprising:
an exosome; and
cargo, located within the exosome, comprising at least one plasmid, wherein the cargo transduces allogenic T cells into CAR-T cells comprising at least one antigenic target.

14. The composition of claim 13, wherein the at least one antigenic target is CD19, CD123, CD33, CD138, NKG2D-L, BCMA, CD5, CD7, CD20, IgKappa, CD22, CD174, IL1RAP, CD30, CD133, ROR1, MIC-A/MIC-B/ULBP, ERBB2, Mesothelin, GD2, EGFR, EDRvIII, EPCAM, MUC1, C-MET, CD171, CD70, Claudin18, GPC3, EPHA2, FAP, IL13RA2, LMP1, MG7, NY-ESO-1, PD-L1, PSCA, CEA, PSMA, VEGFR2, FR-Alpha, or a combination thereof.

15. The composition of claim 14, wherein the cargo further comprises a CRISPR-CAS9 system, a Zinc finger, a single base editor, or a combination thereof.

16. The composition of claim 14, wherein the cargo further comprises siRNA, GalNAc, siRNA-GalNAc, or a combination thereof.

17. The composition of claim 14, wherein the at least one plasmid comprises a promoter.

18. The composition of claim 14, wherein the exosome further comprises at least one targeting agent, protein epitope, or a combination thereof.

19. The composition of claim 14, wherein the exosome is isolated from a cell line, a primary cell culture, or a combination thereof.

20. The composition of claim 14, wherein the exosome is isolated from allogenic cells of a patient.
